(19) United States
(12) Patent Application Publication
(10) Pub. No.: US 2025/0185972 A1
(43) Pub. Date: Jun. 12, 2025

(54) BODY-ATTACHED ELECTROMYOGRAM SENSOR

(71) Applicant: DAEGU GYEONGBUK INSTITUTE OF SCIENCE AND TECHNOLOGY, Daegu (KR)

(72) Inventors: Sang Hoon LEE, Daegu (KR); Jaeu PARK, Daegu (KR); Jin Woong JEONG, Sejong (KR)

(73) Assignee: DAEGU GYEONGBUK INSTITUTE OF SCIENCE AND TECHNOLOGY, Daegu (KR)

(21) Appl. No.: 18/845,176

(22) PCT Filed: Apr. 7, 2023
(86) PCT No.: PCT/KR2023/004737

§ 371 Date: Sep. 9, 2024

(30) Foreign Application Priority Data
Aug. 17, 2022 (KR) 10-2022-0102491

(51) Int. Cl. A61B 5/296; A61B 5/251

(52) U.S. Cl. CPC A61B 5/296; A61B 5/251

(57) ABSTRACT

The present invention relates to a body-attached electromyogram sensor that detects signals from a muscle and provides the degree of contraction and relaxation of the muscle. More specifically, the present invention relates to a body-attached electromyogram sensor that is formed very thin so that the electromyogram sensor may be attached more naturally inside a socket physically connecting a robot leg of an amputee, and thus is more comfortable to wear. In addition, the body-attached electromyogram sensor is made of a material that is elastic and thus naturally expands and contracts according to the movement of the muscle, and is breathable and thus may effectively discharge secretions such as sweat. Accordingly, the body-attached electromyogram sensor may be worn repeatedly and attached for long periods of time.

### TECHNICAL FIELD

The present invention relates to a body-attached electromyogram sensor that detects signals from a muscle, and more particularly, to a body-attached electromyogram sensor that may be used for long periods of time by being attached inside a silicone liner and a socket used by an amputee.

### BACKGROUND ART

An electromyogram sensor is a device that detects signals from a muscle and may identify degrees of contraction and relaxation of the muscle. Information acquired through the electromyogram sensor may be used for technology to control bionic limbs based on an amputee's intention by being used to analyze a muscle state, and recording the amputee's muscle bio-signals for intention analysis.

Commonly used surface electromyography electrodes may be attached to a skin and measure a synthesized signal of motor unit action potentials that is generated from surrounding muscle fibers, thereby identifying a muscle activation degree. To describe an example of a conventional commercial electromyogram sensor, the commercial electromyogram sensor used in clinical practice may use the electrode including silver (Ag)-Ag/chloride (Cl) and have a disposable sticker form to be easily attached and removed. Here, a metal protrusion may protrude from the back of the electrode, and a snap electrode may be electrically connected to the metal protrusion to thus measure the signal. In addition, the electromyogram sensors used in the clinical practice or research are recommended to measure the muscle activation degree by being attached to the skin at a distance of about 2 cm in an axial direction of a target muscle.

However, the conventional electromyogram sensor is disposable, which is unsuitable to be repeatedly used, and has the simple sticker form, which includes a material that is non-elastic and non-breathable. As a result, the conventional electromyogram sensor may be greatly affected by noise due to a separation between the electrode and the skin, caused by a change in a skin surface from muscle contraction and relaxation. In addition, an attachment part of the electromyogram sensor is unable to absorb or discharge secretions such as sweat occurring from the skin, and its long-term use may thus cause lower performance of the electrode and skin trouble.

In addition, when using a robot leg, the amputee is necessarily required to wear a socket physically connecting the robot leg to an amputation site and a silicone liner for adhesion between the socket and the skin. Here, in order to fix the relatively heavy robot leg, the skin, the silicone liner, and the socket are required to be fixed together while maintaining their very close contact, which may apply a strong pressure to a connection portion of the amputee's body when walking. Therefore, wearing the conventional commercial electromyogram sensor in order to use the technology to control the bionic limbs based on the amputee's intention by identifying the amputee's bio-muscle signals may cause great discomfort for the amputee, and wearing the conventional sensor for long periods of time may also be difficult for the amputee. Accordingly, difficulties and limitations may occur in a process of identifying the amputee's bio-muscle signals by using the conventional electromyography sensor.

### DISCLOSURE

### Technical Problem

An object of the present invention is to provide an electromyogram sensor attached to a human body and detecting signals from a muscle, and more particularly, a body-attached electromyogram sensor including an electrode having the minimum thickness possible to be attached to an amputee's muscle and then enable a socket to be worn over the sensor or used by being attached inside the socket, having elasticity to allow its element to expand based on a change in a skin surface that is caused by a muscle movement, and having adhesiveness and breathability to be attached to the body for long periods of time.

In particular, an object of the present invention is to provide an electromyogram sensor that may effectively identify bio-muscle signals based on a walking intention of an amputee wearing a robot leg, and may be applied to a field of bionic limbs in the long term.

### Technical Solution

In one general aspect, provided is a body-attached electromyogram sensor, including a surface electromyography electrode, the sensor including: the surface electromyography electrode; and a substrate, wherein the surface electromyography electrode includes a first electrode layer of a film disposed on its bottom surface, a metal layer deposited on an upper surface of the first electrode layer, and a second electrode layer deposited on an upper surface of the metal layer, and the substrate is disposed on a lower surface of the surface electromyography electrode and includes at least a porous silicon layer.

The substrate may include a silicone adhesive layer coated on a lower surface of the first electrode layer, and

the porous silicon layer coated on a lower surface of the silicone adhesive layer, and the substrate may have an area larger than that of the surface electromyography electrode.

The at least one pair of surface electromyography electrodes may be disposed within the substrate while being spaced apart from each other by a predetermined distance, and one in the pair of surface electromyography electrodes may be a negative electrode and the other may be a positive electrode.

The surface electromyography electrodes may include a connector connecting the pair of surface electromyography electrodes to each other.

The surface electromyography electrode may have its length in a Y-axis longer than its length in an X-axis, the pair of surface electromyography electrodes may be spaced apart from each other in an X-axis direction, and a distance between respective centers of the surface electromyography electrodes may be within 18 to 20 mm.

An electrical lead for each surface electromyography electrode may be connected to the connector.

The porous silicon layer may include at least one material selected from the group consisting of polyimide (PI), polyurethane (PU), styrene butadiene styrene (SBS), styrene ethylene butylene styrene (SEBS), polystyrene (PS), polycaprolactone (PCL), polyacrylonitrile (PAN), polymethyl methacrylate (PMMA), polyvinylidene fluoride (PVDF), polyvinyl chloride (PVC), nylon, polydimethylsiloxane (PDMS), shape memory polymer (SMP), and ecoflex silicone rubber.

The metal layer may include a first metal layer that is an adhesion layer film deposited on the upper surface of the first electrode layer, and a second metal layer that is a conduction layer deposited on an upper surface of the first metal layer, and each of the first metal layer and the second metal layer may include at least one material among titanium (Ti), chromium (Cr), gold (Au), silver (Ag), copper (Cu), molybdenum (Mo), and high-conductivity polymer (including poly(3,4-ethylenedioxythiophene) (PEDOT) and poly(styrenesulfonate) (PSS)).

Each of the first electrode layer and the second electrode layer may include at least one material selected from the group consisting of polyimide, polycaprolactone (PCL), shape memory polymer (SMP), and parylene (parylene C).

The surface electromyography electrode may be serpentine.

The body-attached electromyogram sensor may have a thickness of 70 to 370 μm that includes a thickness of the surface electromyography electrode and a thickness of the substrate.

### Advantageous Effects

The body-attached electromyogram sensor of the present invention configured as described above may be suitable for use by being attached to the body for long periods of time by having the minimum thickness possible to be in close contact with the skin, and having the elasticity and the breathability to be easily attached to the body, may be attached with less discomfort particularly when used by being attached inside the socket of the amputee, and may be free from the secretions such as sweat occurring from the body during its use for long periods of time to thus receive more stable muscle signals.

In addition, the body-attached electromyogram sensors may respectively be attached to the tibialis anterior and gastrocnemius muscles, and record the muscle signals simultaneously, thus enabling the simultaneous recording of the multiple channels. In addition, compared to the conventional commercial product, the body-attached electromyogram sensor may have less interference in the signal recording because the gastrocnemius muscle signal ratio (based on the electrode attached to the tibialis anterior muscle) is lower than the tibialis anterior muscle signal in the specific movement, and may thus acquire the more accurate muscle signal information and use the same for the robot operation.

### DESCRIPTION OF DRAWINGS

FIG. 1 is a cross-sectional view of a body-attached electromyogram sensor.

FIG. 2 is a front view of the body-attached electromyogram sensor.

FIG. 3 shows examples of surface electromyography electrodes having different contact areas.

FIG. 4 is a graph showing resistance change due to a tensile force along an X-axis and a Y-axis based on each contact area of FIG. 3.

FIG. 5 is a front view of the manufactured body-attached electromyogram sensor.

FIG. 6 is a daily water vapor transmission rate (WVRT) of the body-attached electromyogram sensor.

FIG. 7 is a graph showing a mechanical feature of a substrate.

FIG. 8 is a configuration diagram of the body-attached electromyogram sensor.

FIG. 9 is a detailed configuration diagram of the body-attached electromyogram sensor according to an embodiment.

FIG. 10 is an example of a ground electrode.

FIG. 11 is a graph showing resistance change rate versus strain of the surface electromyography electrode.

FIG. 12 is a graph showing resistance change rate versus repeated deformation cycle of the surface electromyography electrode.

FIG. 13 is a graph showing signal-to-noise ratio (SNR) change over time of the surface electromyography electrode.

FIG. 14 is a graph showing changes in the adhesion and skin-electrode interface impedance of the body-attached electromyogram sensor based on its repeated attachment and detachment.

FIG. 15 is a graph showing muscle signal measurement of the body-attached electromyogram sensor at a tibialis anterior (TA) muscle and a gastrocnemius (GC) muscle.

FIG. 16 is a graph showing muscle signal measurement of the body-attached electromyogram sensor at the tibialis anterior (TA) muscle and the gastrocnemius (GC) muscle in various walking environments.

FIG. 17 is an embodiment where the body-attached electromyography sensor is attached.

### BEST MODE

Hereinafter, a technical spirit of the present invention is described in more detail with reference to the accompanying drawings. Prior to the description, terms and words used in the specification and claims are not to be construed as general or dictionary meanings, and are to be construed as meaning and concepts meeting the spirit of the present invention based on a principle that the present inventors may appropriately define the concepts of terms in order to describe their inventions in the best mode.

Therefore, configurations described in the embodiments and accompanying drawings of the present invention do not represent all of the technical spirits of the present invention, and are merely most preferable embodiments. Therefore, the present invention should be construed as including all the changes and substitutions included in the spirit and scope of the present invention at the time of filing this application.

Hereinafter, the spirit of the present invention is described in more detail with reference to the accompanying drawings. The accompanying drawings are only examples shown in order to describe the spirit of the present invention in more detail. Therefore, the spirit of the present invention is not limited to forms of the accompanying drawings.

The present invention provides a body-attached electromyogram sensor 1000 that records muscle bio-signals and may use the same for a clinical purpose. The sensor 1000 may have elasticity to be relaxed with a muscle movement and detect signals from a muscle when attached to the muscle to be identified, and may have breathability to discharge secretions such as sweat occurring from an amputee body when used by being attached to the body for long periods of time. In addition, the body-attached electromyogram sensor 1000 of the present invention may include a surface electromyography electrode 100 having a very small thickness to thus be particularly attached between a socket, which is worn to connect a robot leg of an amputee to an amputation site, and a silicone liner, which is worn to fix the socket to an amputee skin, while maintaining very close contact of the socket with the skin, thereby minimizing irritation in use and improving wearing comfort by being attached more naturally inside the socket.

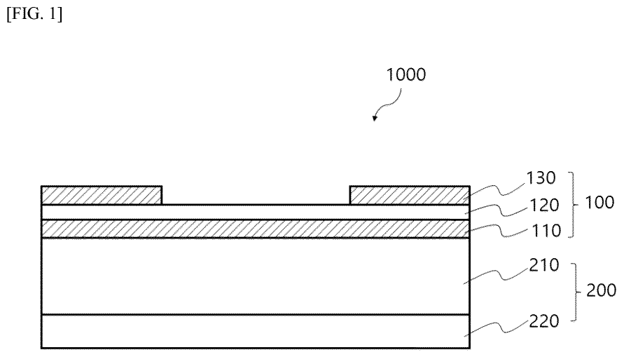

Here, to describe with reference to FIG. 1, the body-attached electromyogram sensor 1000 of the present invention including the surface electromyography electrode 100 may include: the surface electromyography electrode 100; and a substrate 200, wherein the surface electromyography electrode 100 includes a first electrode layer 110 of a film disposed on its bottom surface, a metal layer 120 deposited on an upper surface of the first electrode layer 110, and a second electrode layer 130 deposited on an upper surface of the metal layer 120, and the substrate 200 is disposed on a lower surface of the surface electromyography electrode 100 and includes at least a porous silicon layer 220.

The body-attached electromyogram sensor 1000 of the present invention may include the surface electromyography electrode 100 and the substrate 200, and the substrate 200 may be attached to the body and the muscle bio-signal may be received through the surface electromyography electrode 100. Here, the substrate 200 may include at least the porous silicon layer 220 to thus have the elasticity and the breathability to discharge impurities such as sweat occurring from the body when the sensor 1000 is used for the long periods of time while being attached to the body.

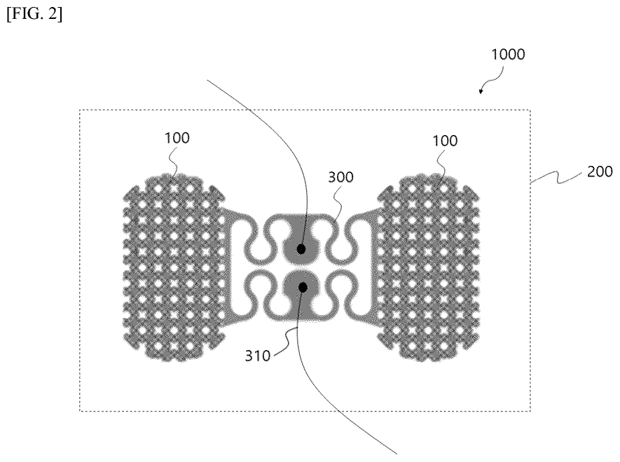

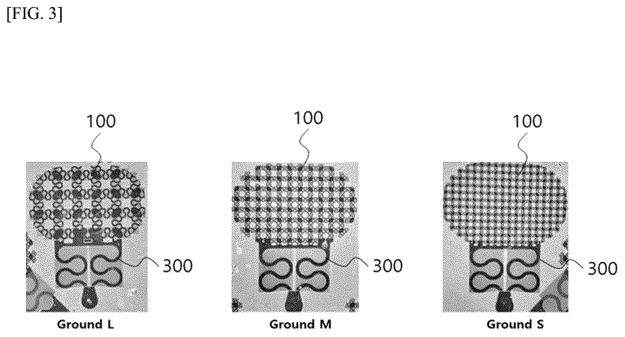

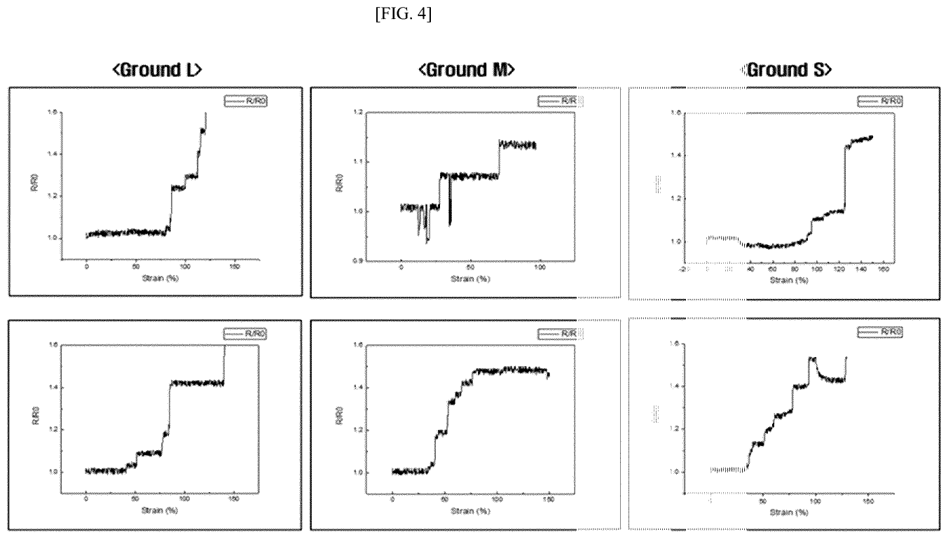

To describe in more detail with reference to FIGS. 1 and 2, the surface electromyography electrode 100 may be formed as a stable, micro-patternable electrode having excellent biocompatibility for acquiring bio-muscle signals. Accordingly, the surface electromyography electrode 100 may be formed by depositing the first electrode layer 110, the metal layer 120, and the second electrode layer 130, and have a predetermined pattern to be stably changed based on the expansion and contraction of a skin surface. According to an embodiment of the present invention, the surface electromyography electrode 100 may preferably be designed to have a serpentine pattern to efficiently disperse a mechanical stress, and change its performance by adjusting pattern density. Here, as shown in FIG. 3, an electrode contact area may be determined by adjusting the pattern density. In addition, as shown in FIG. 4, resistance change may occur due to respective tensile forces of the surface electromyography electrodes, which have different contact areas respectively formed along an X-axis and a Y-axis (in a graph of FIG. 4, the X-axis shows a degree of strain (%) and the Y-axis shows a degree of R/R0). FIG. 3 shows that Ground L has an electrode contact area of 68.33 mm², Ground M has an electrode contact area of 63.88 mm², and Ground S has an electrode contact area of 60.30 mm². The sensor 1000 of the present invention may maintain a resistance change rate to 10% or less even when a deformation of 1.5 times or more occurs because the surface electromyography electrode 100 has the serpentine pattern.

In addition, to describe with reference to FIGS. 1 and 2, the surface electromyography electrode 100 of the present invention may have the minimum thickness by having the first electrode layer 110, the metal layer 120, and the second electrode layer, each of which is formed in the serpentine pattern, and thus be worn more closely between the body and the socket, particularly when attached inside the socket of the amputee. Accordingly, each of the first electrode layer 110 and the second electrode layer 130 may preferably use a material that is stable, may be finely patterned while being formed in the serpentine pattern, and as an example of the present invention, each of the first electrode layer 110 and the second electrode layer 130 may use a flexible and elastic biocompatible material including polyimide. In addition, the metal layer 120 may preferably use a metal material that is electrochemically safe, has a long lifespan, is independent of a composition of an electrolyte, and may prevent contamination of the signals due to the secretions such as sweat. Here, the metal layer 120 may include a first metal layer that is a film deposited on the upper surface of the first electrode layer 110, and a second metal layer deposited on an upper surface of the first metal layer. The first metal layer may be an adhesion layer, which is a thin film for increasing adhesion between the first electrode layer 110 and the second metal layer. As an example of the present invention, the first metal layer may use a material including titanium (Ti). In addition, the second metal layer may be a conduction layer that functions to record an actual muscle signal, and use a material including gold (Au). The second metal layer may have a thickness of about 50 to 1000 nm.

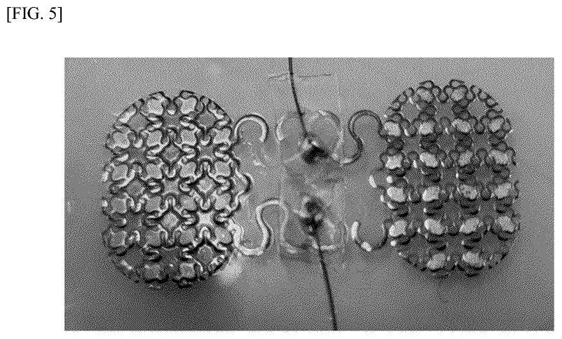

To describe with reference to FIG. 5, the body-attached electromyogram sensor 1000 of the present invention may be formed by coating the substrate 200 including the porous silicon layer 220 on the lower surface of the surface electromyography electrode 100. The body-attached electromyogram sensor 1000 of the present invention may have a multilayer structure using the flexible, elastic, and breathable substrate 200 which may expand and contract based on a muscle surface movement to enable its repeated and long-term attachment to the skin. Accordingly, the substrate 200 may include an elastic and breathable silicone adhesive layer 210 coated on a lower surface of the first electrode layer 110, and the flexible and elastic porous silicon layer 220 coated on a lower surface of the silicone adhesive layer 210. The porous silicon layer 220 may be used for attaching the surface electromyography electrode 100 of the present invention to the skin. The porous silicon layer 220 may preferably have the serpentine pattern, and use an elastic material in order for the surface electromyography electrode 100, whose shape is changed based on a skin movement, to maintain its shape and attachment to the skin. The porous silicon layer 220 may include at least a polydimethylsiloxane (PDMS) material, and particularly use phosphorus PDMS (pPDMS) that may form more porous pores. However, the porous silicon layer 220 may be replaced by at least one selected from the group consisting of polyimide (PI), polyurethane (PU), styrene butadiene styrene (SBS), styrene ethylene butylene styrene (SEBS), polystyrene (PS), polycaprolactone (PCL), polyacrylonitrile (PAN), polymethyl methacrylate (PMMA), polyvinylidene fluoride (PVDF), polyvinyl chloride (PVC), nylon, polydimethylsiloxane (PDMS), shape memory polymer (SMP), and ecoflex. The silicone adhesive layer 210 may preferably include a material that uses an adhesive material to connect the first electrode layer 110 and the porous silicon layer 220 to each other, and also has the elasticity while securing the breathability to discharge the secretions such as sweat, and may use silbione as an example. Accordingly, the body-attached electromyogram sensor 1000 of the present invention may provide a sensor that may be attached between the socket and the silicone liner for fixing the robot leg to the amputee body with the minimum irritation, is adhesive to thus enable its repeated attachment, and has the breathability to thus allow a smooth water vapor transmission rate from the impurities such as sweat that may occur from the body due to its long-term wear.

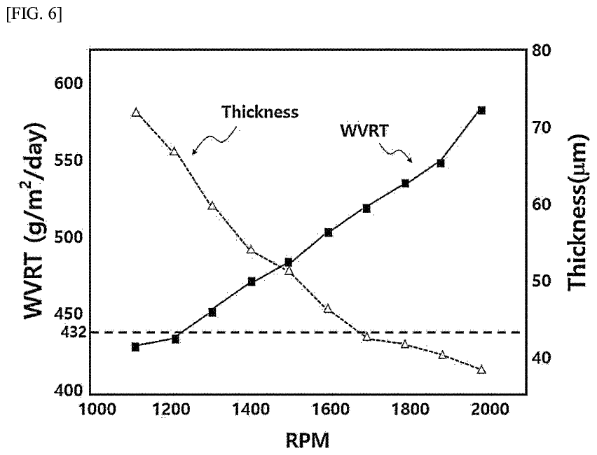

FIG. 6 shows an experiment on water vapor transmission rate, and a dotted line shows the average amount of a daily water vapor transmission rate exhaled by a person. Referring to FIG. 6, it may be seen that the porous silicon layer 220 implements a daily water vapor transmission rate (WVTR) of the body-attached electromyogram sensor 1000 in the present invention of about 450 g/m²/day, which is higher than a daily water vapor transmission rate of 432 g/m²/day exhaled by a person that is suggested in a document. Accordingly, the body-attached electromyogram sensor may allow its close contact with the amputation site and comfortable use by the amputee even when worn for the long periods of time.

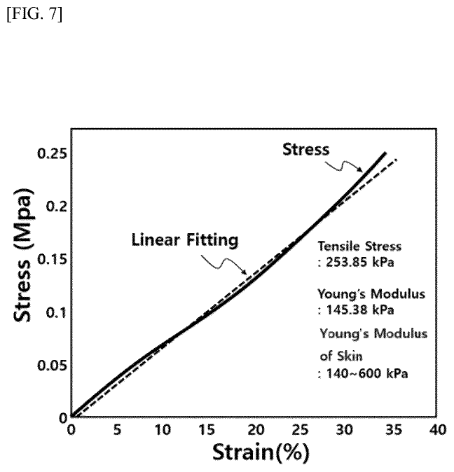

The body-attached electromyogram sensor 1000 of the present invention may be a sensor having the minimum thickness including a thickness of the surface electromyography electrode 100 and a thickness of the substrate 200 to thus be attached more naturally inside the socket connecting the robot leg of the amputee to the amputation site, thereby improving the amputee's wearing comfort. Accordingly, the thickness of the sensor in the present invention that includes the thickness of the surface electromyography electrode 100 and the thickness of the substrate 200 may preferably be 220±150 μm, and particularly 350 μm. Here, the silicone adhesive layer 210 may have a thickness of 30 to 280 μm, more specifically, about 270 μm, and the porous silicon layer 220 may have a thickness of 40 to 80 μm, more specifically, about 70 μm. Therefore, FIG. 7 shows a tensile test on the substrate. To describe with reference to FIG. 7, Young's Modulus of the body-attached electromyogram sensor 1000 of the present invention may be implemented as about 150 kPa, which is a similar range to Young's Modulus of the skin, ranging from 140 to 600 kPa, suggested in the document, by the substrate 200, thus enabling close contact of the sensor for various muscle uses.

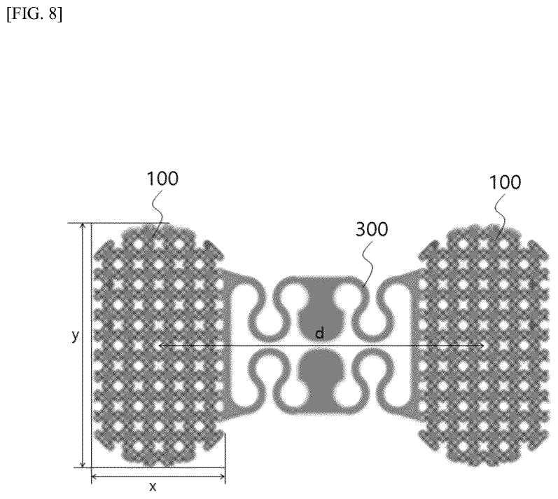

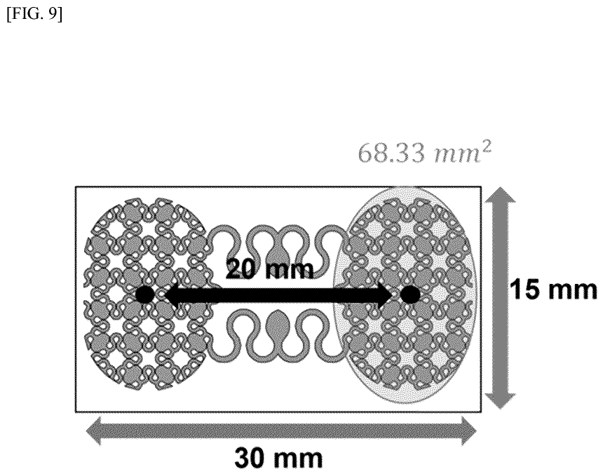

In addition, the body-attached electromyogram sensor 1000 of the present invention may have the form of an electrode array where one or more surface electromyography electrodes 100 are disposed in the substrate 200, as needed. The surface electromyography electrode 100 of the present invention may be manufactured to have its size and configuration in accordance with the configuration and design guidelines of the sensor that are recommended by Surface ElectroMyoGraphy for the Non-Invasive Assessment of Muscles (SENIAM). To describe with reference to FIGS. 5 and 8, in an embodiment of the present invention, the pair of surface electromyography electrodes 100 may be disposed to be parallel to each other within the substrate 200 of the body-attached electromyogram sensor 1000, and this pair of electrodes may be connected to each other by a connector 300. In more detail, in the body-attached electromyogram sensor 1000 of the present invention, the substrate 200 may be larger than the surface electromyography electrode 100. In addition, the substrate 200 may be suitably sized to accommodate the plurality of surface electromyography electrodes 100, and the pair of surface electromyography electrodes 100 may be disposed within the substrate 200 to be spaced apart from each other. The connector 300 may be serpentine and connect the pair of surface electromyography electrodes 100. Here, serpentine density of the connector 300 may preferably be greater than serpentine density of the surface electromyography electrode 100. In the present invention, the pair of surface electromyography electrodes 100 within the substrate 200 may include its negative and positive electrode. One of the pair of surface electromyography electrodes 100 may be configured to detect the signal from a moving muscle, and the other may be configured as a reference signal. Therefore, the signal may be received using a differential amplifier by differentially recording each signal for the muscle to which the sensor is attached. Accordingly, the body-attached electromyogram sensor 1000 of the present invention may introduce a differential configuration that eliminates external noise by using all the two electrodes, and may be easily connected to an external amplifier. In addition, the connector 300 may connect the pair of surface electromyography electrodes 100 including the negative electrode and the positive electrode to each other, and an electrical lead 310 may be disposed at the connector 300 to receive the bio-muscle signals. The electrical lead 310 may be stably soldered by being selected as the electrical lead 310 (e.g., 36 AWG) that is very thin and has an insignificant effect on neural recording in order to minimize the irritation that the amputee may feel after the body-attached electromyogram sensor 1000 of the present invention is inserted into the silicone liner connecting the robot leg to the amputation site. The electrical lead 310 may be coated with the porous silicon layer 220 of the substrate 200 to thus secure its mechanical stability and electrical insulation. In addition, each of the pair of surface electromyography electrodes 100 may have a rectangular shape in which its length in the Y-axis is longer than its length in the X-axis, and the pair of surface electromyography electrodes 100 may be spaced apart from each other in an X-axis direction. Here, to describe with reference to FIGS. 8 and 9, each surface electromyography electrode 100 may have a size of about 1 cm, and more specifically, its length in the X-axis may be 8 mm, and its length in the Y-axis may be 11 mm. In addition, a distance between the pair of electrodes may be within 18 to 20 mm. For example, the distance between the respective centers of the pair of electrodes, that is, the distance between their respective centers may be 2 cm. The pair of surface electromyography electrodes 100 may be disposed transversely or longitudinally based on a muscle fiber.

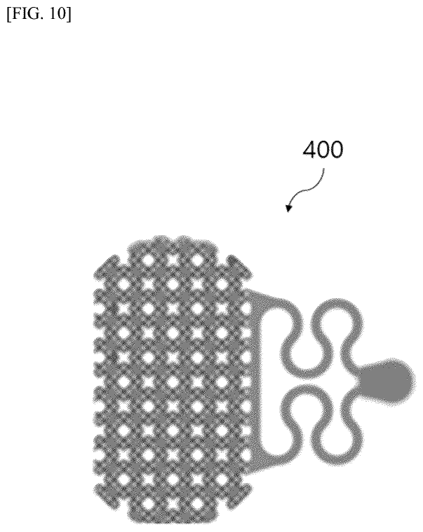

In addition, the body-attached electromyogram sensor 1000 may include a separate ground electrode 400. To describe with reference to FIG. 10, similar to the surface electromyography electrode 100, the ground electrode 400 may include the first electrode layer 110, the metal layer 120, and the second electrode layer 130, and used by being attached to the skin by coating the substrate 200 on the lower surface of the first electrode layer 110. The above ground electrode 400 may be manufactured to have the serpentine pattern like the surface electromyography electrode 100, have a size similar to that of the surface electromyography electrode 100, and be replaced by another conventional electrode, as needed. It may be more efficient for the ground electrode 400 to be spaced apart by a predetermined distance from the surface electromyography electrode 100 to be detected.

To briefly describe a manufacturing process of the body-attached electromyogram sensor 1000 of the present invention, the surface electromyography electrode 100 may be formed by first depositing the first electrode layer 110, the metal layer 120, and the second electrode layer 130 sequentially from its bottom surface. Here, in order for the surface electromyography electrode 100 to have the serpentine pattern, light of a short wavelength may be irradiated onto a photosensitive polymer applied onto a wafer by using a film mask on which a design of the first electrode layer 110 to be patterned is drawn, and the photosensitive polymer may then be patterned using a developer. In addition, a photosensitive photoresist may then be applied on the photosensitive polymer, and the photoresist may then be formed into a desired pattern by irradiating light onto the photoresist by using a film mask on which the metal layer 120 is drawn. The adhesion layer of the metal layer and a metal film of the conduction layer may then be deposited on the photoresist, and the patterned photoresist may be removed. In this way, the surface electromyography electrode 100 may have a desired serpentine electrode pattern. In addition, the second electrode layer 130 may also be patterned in the same method as the first electrode layer 110 by using a photolithography method to form the surface electromyography electrode. A sacrificial layer may be additionally formed for the surface electromyography electrode 100 to maintain its shape even after being separated from the wafer. The photosensitive photoresist may be applied on the surface electromyography electrode 100, and the sacrificial layer may then be formed using the photolithography method. In order to manufacture the body-attached electromyogram sensor 1000, the wafer and the surface electromyography electrodes may be separated from each other, the surface electromyography electrode 100 manufactured first may then be transferred to the substrate 200 including the silicone adhesive layer 210 and the porous silicon layer 220, and the sacrificial layer may then be removed using acetone or a remover. The electrical lead 310 may then be fixed to the connector 300 by using a conductive paste (e.g., silver paste) or lead, and their connection part may be encapsulated using epoxy (e.g., ultraviolet (UV) epoxy) or the flexible and elastic biocompatible material. Finally, the film (e.g., polyethylene terephthalate (PET)) and the device may be separated from each other to thus manufacture the body-attached electromyogram sensor 1000.

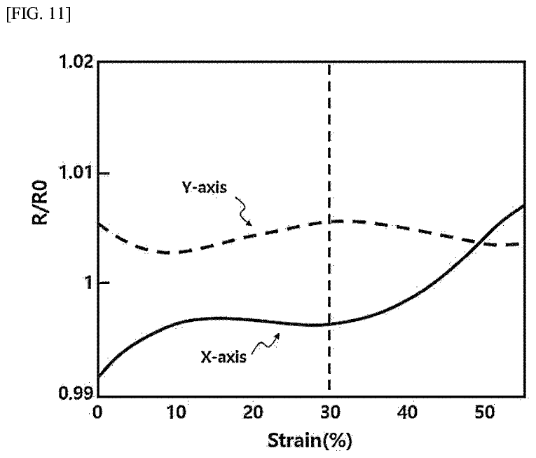

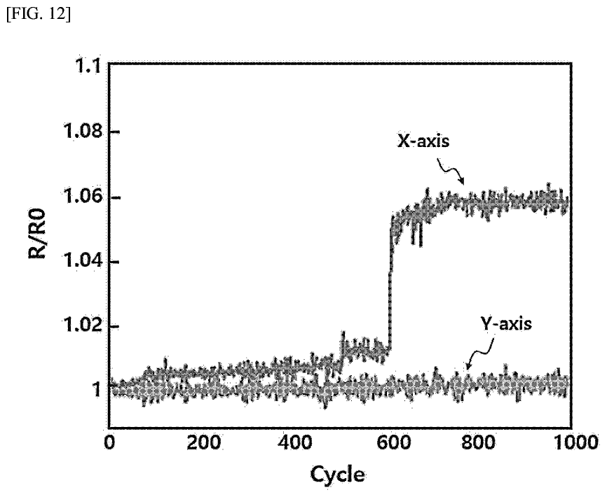

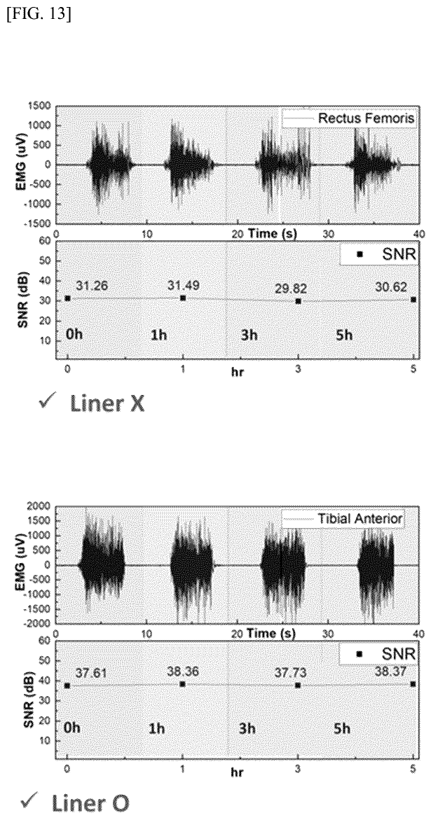

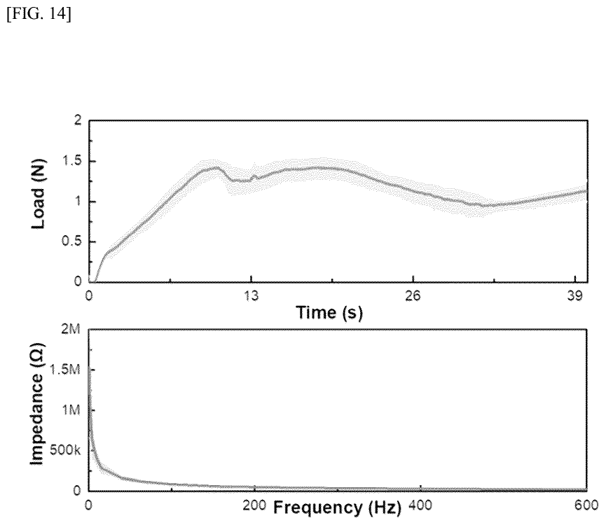

The present invention provides the body-attached electromyogram sensor 1000 including the serpentine surface electromyography electrode 100 that has the elasticity and durability along both the X and Y axes. Referring to FIG. 11, it may be seen that the resistance change rate is less than 1% even when the surface electromyography electrode 100 of the present invention is deformed by 55%. Referring to FIG. 12, it may be seen that the resistance change rate is less than 10% even after the surface electromyography electrode is deformed by 30% and repeating this deformation 1000 times. This result shows that the electrical and mechanical features of the surface electromyography electrode 100 are well preserved even under skin perturbation due to the muscle contraction when considering that a human skin strain is 30% in the document. In addition, as shown in FIG. 13, signal-to-noise ratio (SNR) change is observed over time to observe changes in the adhesion and signal acquisition ability of the body-attached electromyogram sensor 1000 that occur due to its long-term use. The adhesion and signal acquisition ability of the sensor 1000 is measured while the liner is worn to provide an environment similar to that used by the amputee, and it may be seen that the SNR change is very insignificant. FIG. 14 shows the changes in the adhesion and skin-electrode interface impedance of the body-attached electromyogram sensor 1000 based on its repeated attachment and detachment, and it may be seen that these changes are very insignificant. These insignificant changes indicate that the proposed body-attached electromyogram sensor 1000 may maintain its performance after its long-term use.

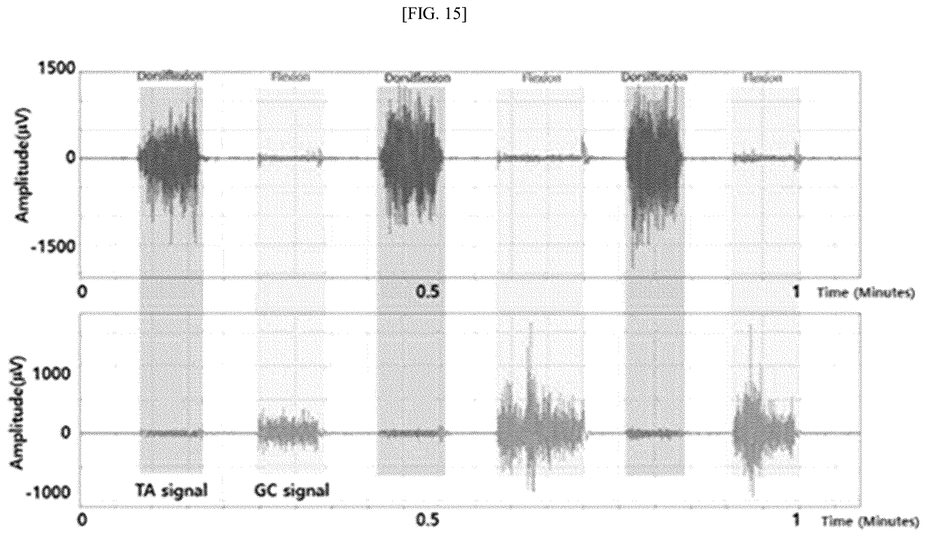

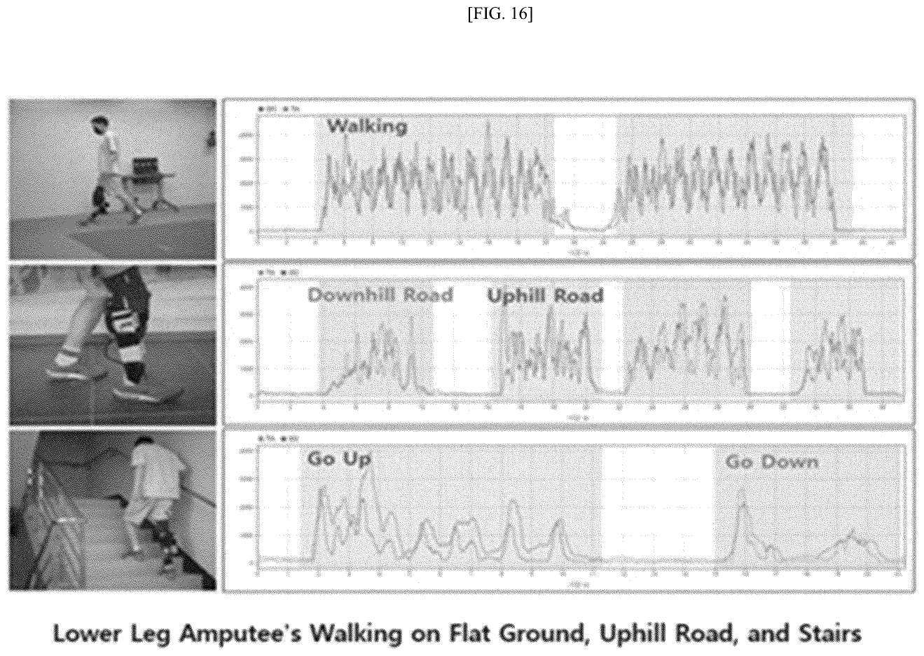

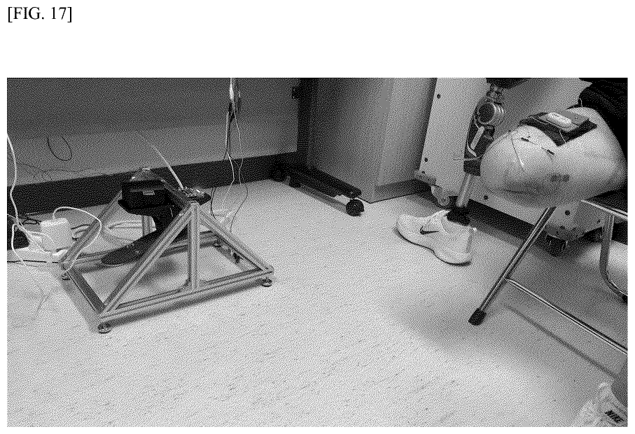

The present invention may include the plurality of body-attached electromyogram sensors 1000 each including the pair of surface electromyography electrodes 100. One or more body-attached electromyogram sensors 1000 may respectively be attached to the tibialis anterior and gastrocnemius muscles (TA and GC muscles or GC and TA muscles) to thus simultaneously record the muscle signals based on a muscle flexion movement such as ankle flexion or dorsiflexion. In FIG. 15, the performance of the surface electromyography electrode 100 of the present invention is compared with that of a commercial product. In the muscle signals based on the flexion movement of the dorsiflexion muscle, the average root mean square (rms) of the GC signals is 16.72 uV, the average rms of the TA signals is 26.302 uV, and the average rms of noise is 5.35 uV. In addition, it is observed that the signal-to-noise ratio (SNR) of the GC signal is 9.89 dB, which is lower than the SNR (17 dB) of the commercial product, and the SNR of the TA signal is 33.83 dB, which is lower than the SNR (42 dB) of the commercial product. In addition, in the muscle signals based on the flexion movement of the ankle flexion muscle, the average rms of the TA signals is 18.97 uV, the average rms of the GC signals is 120.53 uV, and the average rms of noise is 5.95 uV. In addition, the SNR of the TA signal is 10.07 dB, which is lower than the SNR (25 dB) of the commercial product, and the SNR of the GC signal may be 26.13 dB, which is lower than the SNR (39 dB) of the commercial product. The SNR values in parentheses of FIG. 15 show the performance of the commercial product. Accordingly, it may be seen that the overall SNR is similar to or slightly lower than that of the commercial product, and there is less signal interference than the commercial product in the simultaneous measurement of the TA muscle and the GC muscle. That is, when the body-attached electromyogram sensor 1000 attached to one muscle is activated, the body-attached electromyogram sensor 1000 attached to another muscle may detect the signal having a relatively low intensity. Therefore, the signals detected by the sensors attached to the respective muscles may have less interference, thus separately receiving the signals for the movement of the respective muscles. FIG. 16 is a graph showing each muscle signal for a lower leg amputee's walking on flat ground, an uphill road, and stairs, and FIG. 17 is an embodiment where the body-attached electromyogram sensor of the present invention is attached to the amputee. Accordingly, referring to FIGS. 16 and 17, compared to a commercial sensor, the body-attached electromyogram sensor 1000 of the present invention may stably and effectively acquire the bio-muscle signals based on a walking intention of the amputee wearing the robot leg, and minimize amputee's discomfort in using the sensor by attaching the same to the body for the long periods of time. Therefore, it is expected that the sensor 1000 is applied to a field of bionic limbs in a long term.

Hereinabove, although the present invention has been described by specific matters such as the detailed components, the embodiments and the accompanying drawings are provided only for assisting in comprehensive understanding of the present invention. Therefore, the present invention is not limited to the embodiments, and various modifications and changes may be made by those skilled in the art to which the present invention pertains from this description.

Therefore, the spirit of the present invention should not be limited to the embodiments described above, and the following claims and all of modifications equal or equivalent to the claims are intended to fall within the scope and spirit of the present invention.

<Description of symbols>

```
1000: body-attached electromyogram sensor
  100: surface electromyography electrode
  110: first electrode layer
  120: metal layer
  130: second electrode layer
  200: substrate
  210: silicone adhesive layer
  220: porous silicon layer
  300: connector
  310: electrical lead
  400: ground electrode
```

1. A body-attached electromyogram sensor including a surface electromyography electrode, comprising:
the surface electromyography electrode; and
a substrate,
wherein the surface electromyography electrode includes
a first electrode layer of a film disposed on its bottom surface,
a metal layer deposited on an upper surface of the first electrode layer, and
a second electrode layer deposited on an upper surface of the metal layer, and
the substrate is disposed on a lower surface of the surface electromyography electrode and includes at least a porous silicon layer.

2. The sensor of claim 1, wherein the substrate includes
a silicone adhesive layer coated on a lower surface of the first electrode layer, and
the porous silicon layer coated on a lower surface of the silicone adhesive layer, and
the substrate has an area larger than that of the surface electromyography electrode.

3. The sensor of claim 2, wherein the at least one pair of surface electromyography electrodes are disposed within the substrate while being spaced apart from each other by a predetermined distance, and
one in the pair of surface electromyography electrodes is a negative electrode and the other is a positive electrode.

4. The sensor of claim 3, wherein the surface electromyography electrodes include a connector connecting the pair of surface electromyography electrodes to each other.

5. The sensor of claim 4, wherein the surface electromyography electrode has its length in a Y-axis longer than its length in an X-axis,
the pair of surface electromyography electrodes are spaced apart from each other in an X-axis direction, and
a distance between respective centers of the surface electromyography electrodes is within 18 to 20 mm.

6. The sensor of claim 4, wherein an electrical lead for each surface electromyography electrode is connected to the connector.

7. The sensor of claim 1, wherein the porous silicon layer includes at least one material selected from the group consisting of polyimide (PI), polyurethane (PU), styrene butadiene styrene (SBS), styrene ethylene butylene styrene (SEBS), polystyrene (PS), polycaprolactone (PCL), polyacrylonitrile (PAN), polymethyl methacrylate (PMMA), polyvinylidene fluoride (PVDF), polyvinyl chloride (PVC), nylon, polydimethylsiloxane (PDMS), shape memory polymer (SMP), and ecoflex.

8. The sensor of claim 1, wherein the metal layer includes
a first metal layer that is an adhesion layer film deposited on the upper surface of the first electrode layer, and
a second metal layer that is a conduction layer deposited on an upper surface of the first metal layer, and
each of the first metal layer and the second metal layer includes at least one material among titanium (Ti), chromium (Cr), gold (Au), silver (Ag), copper (Cu), molybdenum (Mo), and high-conductivity polymer (including poly(3,4-ethylenedioxythiophene) (PEDOT) and poly(styrenesulfonate) (PSS)).

9. The sensor of claim 1, wherein each of the first electrode layer and the second electrode layer includes at least one material selected from the group consisting of polyimide, polycaprolactone (PCL), shape memory polymer (SMP), and parylene (Parylene C).

10. The sensor of claim 8, wherein the surface electromyography electrode is serpentine.

11. The sensor of claim 1, wherein the body-attached electromyogram sensor has a thickness of 70 to 370 μm that includes a thickness of the surface electromyography electrode and a thickness of the substrate.

12. The sensor of claim 9, wherein the surface electromyography electrode is serpentine.
